(19) United States
(12) Patent Application Publication
(10) Pub. No.: US 2010/0120711 A1
(43) Pub. Date: May 13, 2010

(54) AMPK POTENTIATOR CONTAINING CHITO-OLIGOSACCHARIDE

(75) Inventors: Dae Bang Seo, Gyeonggi-do (KR); Jong Hee Sohn, Seoul (KR); Eui Seok Shin, Gyeonggi-do (KR); Wan Gi Kim, Gyeonggi-do (KR); Nam Hoon Cho, Gyeonggi-do (KR); Myung Hwa Lee, Seoul (KR); Sang Jun Lee, Gyeonggi-do (KR)

Correspondence Address: NIXON & VANDERHYE, PC, 901 NORTH GLEBE ROAD, 11TH FLOOR, ARLINGTON, VA, 22203, US

(73) Assignee: AMOREPACIFIC CORPORATION, Seoul (KR)

(21) Appl. No.: 12/515,928

(22) PCT Filed: Oct. 23, 2007
(86) PCT No.: PCT/KR07/05221

§ 371 Date: Jan. 6, 2010

(30) Foreign Application Priority Data
Nov. 30, 2006 (KR) 10-2006-0119449

(51) Int. Cl. A61K 31/722; A61P 3/00

(52) U.S. Cl. 514/55

(57) ABSTRACT

Disclosed herein is a composition for promoting AMPK activity, which comprises a chito-oligosaccharide as an active ingredient. The present composition affects the enzymes related to AMPK and lipid metabolism, and thus helps recovery from the condition lacking energy by various functions of controlling metabolism. Therefore, the present composition can enhance energy metabolism, thus making the composition effective for improving exercise capacity and reducing fatigue. The present composition can be used in the preparation of food or medicinal product as an endurance builder or for the prevention and improvement of fatigue.

### TECHNICAL FIELD

The present invention relates to a composition for promoting AMP-activated protein kinase (AMPK) activity, which comprises a chito-oligosaccharide as an active ingredient. More specifically, the present invention relates to a composition for enhancing energy metabolism in liver cells by the activation of AMPK and lipid metabolism-related enzymes which comprises a chito-oligosaccharide.

### BACKGROUND ART

Chitosan is broadly used in various fields such as a coagulator for waste water, an absorbent for heavy metals, a functional food, an ion-exchanger, a medicinal product, etc. Recently, it was known that chitin, chitosan and their derivatives exhibit diverse physiological activities such as decholesterol action, anti-cancer effect, inhibition of the increase of blood pressure, control of glucose in blood, improvement of liver function, excretion of heavy metals and contaminants out of the human body, etc., and thus many studies have been made on the substances as a prospective material having a high value added in the field of bio-medical science.

Chito-oligosaccharide, which is a low molecular polysaccharide hydrolyzed from chitosan by an acid or an enzyme, has an absorption in the human body higher than chitosan, and the actions of immune potentiation, anti-oxidation (Shon Y et al., J. Chitin and chitosan, 2001, 6, 107-110) and growth inhibition of cancer cells (Nam M Y, J. Chitin and Chitosan, 1999, 4, 184-188) by a chito-oligosaccharide have been studied. Also, it was reported that chito-oligosaccharide has a function to inhibit the liver damage caused by carbon tetrachloride (Cheju J. Life Science, 2(2), 3-10, 1999). Although chito-oligosaccharide is used as a health food or a conventional food in various ways, there are a few patents related to its function. Further, prior patents do not provide the specific process of energy expenditure in connection with an energy metabolism enhancement.

Meanwhile, AMPK is an enzyme which is activated under the condition lacking intracellular energy, helps recovery from the condition lacking energy by various functions of controlling metabolism, and is activated in a stressed condition where the level of AMP is increased over ATP, thereby increasing intracellular energy (ATP) production (Bergeron R et al., Diabetes, 50, 1076-1082, 2001; Winder W W et al., Am J Physiol 277:E1-10, 1997). It was known that AMPK is widely distributed in the human body and plays a role of energy detector in mediating cell adaptation to the change of intracellular nutritional condition and external environment (Hardie D G, Ann Rev Biochem 67, 821-855, 1998; Hardie D G, Eur J Biochem 246, 259-273, 1997).

Recent studies reported that AMPK responds to the change of intracellular energy, and thus plays an important role in carbohydrate and lipid metabolism as a major mediator for energy conversion. The effect of AMPK activation on the metabolism in a liver was also investigated in many studies. Particularly, acetyl-CoA carboxylase (ACC) and 3-hydroxy-3-methylglutryl-CoA reductase (HMGR), enzymes catalyzing a control step which is important in synthesizing fatty acid and sterol, were leading subjects treated in the studies. It was reported that the activation of AMPK by a stress depleting ATP in an isolated liver cell induces HMGR phosphorylation and inhibits the synthesis of fatty acid and sterol (Corton J M et al., Curr Biol 4, 315-324, 1994; Henin N et al., FASEB J 9, 541-546, 1995). ACC in a liver cell is a potent inhibitor of carnithine palmitoyltransferase-1 (CPT-1) which plays a role in the transport of fatty acids into mitochondria and, when ACC is inactivated, the concentration of metabolites of ACC such as malonyl-CoA which largely affects the oxidation of fatty acid becomes lowered (MaGarry J D, Am J Clin Nutr 67(Suppl. 3), 500s-504s, 1998; McGarry J D et al., Eur J Biochem 244, 1-14, 1997). When a representative AMPK activator, AICA-riboside (AICAR) is cultured with a hepatocyte, the oxidation of fatty acid is increased, and the activity of CPT-1 is increased due to a reduction of the concentration of malonyl-CoA. Also, when AMPK is activated by AICAR in an isolated murine adipocyte, the synthesis of lipid is inhibited by the phosphorylation of ACC (Sullivan J E, FEBS Lett 353, 33-36, 1994).

AMPK is involved in the oxidation of the fatty acid derived from exercise (Musi N et al., Biochemical society transactions 31, 161-195, 2002), and the AMPK activated during exercise in a murine skeletal muscle inactivates ACC-2 by phosphorylation and reduces the amount of Malnoyl CoA in the muscle, which makes fatty acids incorporated into mitochondria. From the study on a human skeletal muscle, it was reported that ACC-2 is phosphorylated during exercise (Chen Z et al., Am J Physiol 279, E1202-E1206, 2000) and thus inactivated, thereby increasing the oxidation of fatty acid (Dean D et al., Diabetes, 49(8), 1295-300, 2000).

As disclosed above, AMPK is an enzyme which is activated under the condition lacking intracellular energy and helps recovery from the condition lacking energy by various functions of controlling metabolism. That is, its activity is increased under the condition that intracellular ATP energy is lowered, for example, by exercise, and thus it functions as a “metabolic sensor” which promotes a metabolism and enhances ATP synthesis. Further, since the production of energy is increased by AMPK activation, the increased energy production under the circumstance requiring energy such as exercise or in everyday life makes exercise capacity improved and fatigues reduced. Therefore, AMPK activator can be used in the preparation of food or medicinal product as an endurance builder or for the prevention and improvement of fatigue.

However, as for the promotion of AMPK activity which exhibits various functions and has a wide range of utility, the effect of chito-oligosaccharide has not been known.

### DISCLOSURE

### Technical Problem

The present inventors have studied the effect of chito-oligosaccharide on the AMPK activity and found the effect of chito-oligosaccharide on the enzymes related to AMPK activity and energy metabolism.

Therefore, an object of the present invention is to provide a composition for promoting AMPK activity, which comprises a chito-oligosaccharide, wherein the composition is effective for enhancing energy metabolism and improving fatigue.

### Technical Solution

To achieve the above object, the present invention provides a composition for promoting AMPK activity, which comprises a chito-oligosaccharide as an active ingredient.

Hereinafter, the present invention will be described in more detail.

Chito-oligosaccharide used in the present invention can be obtained through the following steps: isolating and purifying by triturating shells of crab, shrimp, etc., desalting the triturate, removing proteins and eliminating impurities; deacetylation of chitosan; and hydrolysis of chitosan by a chemical degradation using an inorganic acid such as hydrochloric acid, etc., or by an enzymatic degradation using enzymes.

Specifically, in the degradation method using enzyme, chitosan is added to purified water, 2 to 3% hydrochloric acid is added thereto, and the mixture is stirred at 40 to 60° C. to produce a chitosan dispersion comprising a solid content of 5 to 10% and hydrochloric acid. After dissolving it, pH is adjusted to 4 to 6 and cellulose, which is dissolved in purified water as an enzyme for chitosan hydrolysis, is added thereto. Subsequently, it is hydrolyzed for 14 to 20 hours at 40 to 60° C., heat-treated for 30 minutes at 80° C. to inactivate the hydrolysis enzyme, and filtered and dried to obtain chito-oligosaccharide.

The molecular weight of chito-oligosaccharide is changed according to the amount of cellulase added during said process. When the enzyme is added in an amount of 10% of chitosan, chito-oligosaccharide having a molecular weight of 1,000 or less is obtained; when 6%, 1,500 to 2,000; and when 3%, 7,000 to 10,000. In the present invention, 3 to 10% of enzyme, based on the weight of chitosan, was added and chito-oligosaccharide with a molecular weight of 700 to 9,000 was obtained.

It is preferred that chito-oligosaccharide is comprised in an amount of 10 to 90% by weight based on the total weight of the composition. Considering that when the composition is formulated into a tablet or soft capsule, a powder or a functional components can be comprised in an amount of 10 to 60% and when the composition is formulated into a hard capsule, it can be comprised in an amount of 10 to 90%, a functional food for health, which comprises the composition in an amount of 10 to 90%, can be provided.

Thus, the present invention provides a functional food for health which comprises the chito-oligosaccharide prepared according to said process. The functional food for health includes various formulations such as powder, granule, tablet, capsule and drink. The food is preferably formulated into a unit dosage, wherein each dosage comprises additives together with a given effective component. Also, it cab be further mixed with a suitable diluent, carrier or other excipient according to a conventional preparation method.

The functional food for health can comprise, if necessary, one or more additives selected from the following: an extract of grapefruit, apple extract, orange, lemon, pineapple, banana, pear, etc. (any one of concentrated extract or powdered extract can be used); vitamins (water soluble and oil-soluble vitamins such as palmitic acid retinol, riboflavin, pyridoxin, cyanocobalamine, sodium ascorbic acid, nicotinic amide, calcium pantotenic acid, folic acid, biotin, cholecalciferol, choline bitartrate, tocopherol, β-carotine, etc.); flavorant (lemon flavor, orange flavor, strawberry flavor, grapefruit flavor, vanilla essence, etc.); amino acid, nucleic acid and their salts (glutamic acid, sodium glutamate, glycine, alanine, aspariginic acid, sodium asparaginate, inosinic acid, etc.); plant fiber (polydextrose, pectin, xantan rubber, glucomannan, alginic acid, etc.); minerals (sodium chloride, sodium acetate, magnesium sulfate, potassium chloride, magnesium chloride, magnesium carbonate, calcium chloride, bipotassium phosphate, monosodium phosphate, calcium glycerophosphate, sodium ferrous citrate, ferric ammonium citrate, ferric citrate, manganese sulfate, copper sulfate, sodium iodide, potassium solvate, zinc, manganese, copper, iodine, covalt, etc.).

### Advantageous Effects

AMPK activator affects on enzymes related to lipid metabolism and enhances energy metabolism in liver cells, and thus the production of energy can be increased. The increased energy production under the circumstance requiring energy such as exercise or in everyday life makes exercise capacity improved and fatigues reduced. Therefore, AMPK activator can be used in the preparation of food or medicinal product as an endurance builder or for the prevention and improvement of fatigue.

### DESCRIPTION OF DRAWINGS

FIG. 1 is a graph showing the change in the amount of expression of total AMPK and phospho-AMPK protein in liver cells by an administration of chito-oligosaccharide.

FIG. 2 is a graph showing the change in the amount of expression of total ACC and phospho-ACC protein in liver cells by an administration of chito-oligosaccharide.

FIG. 3 is a graph showing the degree of fatty acid oxidation in liver cells by an administration of chito-oligosaccharide.

FIG. 4 is a graph showing the change in the amount of ATP production in liver cells by an administration of chito-oligosaccharide.

FIG. 5 is a graph showing the immobility time in mice after dietary intake of chito-oligosaccharide.

### BEST MODE

Hereinafter, the present invention will be described in further detail with reference to the following examples and test examples. However, the scope of the present invention is not limited to only to these examples.

### Example 1

### Preparation of Soft Capsule

Chito-oligosaccharide, vitamin E, vitamin C, palm oil, vegetable hardened oil, yellow beeswax and lecithin were mixed in a ratio of 80 mg of chito-oligosaccharide, 9 mg of vitamin E, 9 mg of vitamin C, 2 mg of palm oil, 8 mg of vegetable hardened oil, 4 mg of yellow beeswax and 9 mg of lecithin, and a fluid for filling soft capsules was prepared according to a conventional method. As a separate procedure, 66 parts by weight of gelatin, 24 parts by weight of glycerin and 10 parts by weight of sorbitol fluid were used to prepare a soft capsule sheet. The soft capsules were filled with the fluid to contain the present composition in an amount of 400 mg/capsule.

### Example 2

### Preparation of Tablet

Chito-oligosaccharide, vitamin E, vitamin C, galacto-oligosaccharide, lactose and maltose were mixed in a ratio of 80 mg of chito-oligosaccharide, 9 mg of vitamin E, 9 mg of vitamin C, 200 mg of galacto-oligosaccharide, 60 mg of lactose and 140 mg of maltose, the mixture was granulated with a fluidized bed drier and subsequently 6 mg of sugar ester was added. 504 mg of the composition was compressed into tablets according to a conventional method.

### Example 3

### Preparation of Drink

Chito-oligosaccharide, vitamin E, vitamin C, glucose, citric acid and liquid oligosaccharide were mixed in a ratio of 80 mg of chito-oligosaccharide, 9 mg of vitamin E, 9 mg of vitamin C, 10 g of glucose, 0.6 g of citric acid and 25 g of liquid oligosaccharide and subsequently 300 ml of purified water was added. The solution was filled into bottles in an amount of 200 ml/bottle and the filled bottles were sterilized for 4 to 5 seconds at 130° C. so as to prepare drinks.

### Example 4

### Preparation of Granule

Chito-oligosaccharide, vitamin E, vitamin C, anhydrous crystal glucose and starch were mixed in a ratio of 80 mg of chito-oligosaccharide, 9 mg of vitamin E, 9 mg of vitamin C, 250 mg of anhydrous crystal glucose and 550 mg of starch, the mixture was formulated into granules with a fluidized bed granulator, and the granules were filled into each pack.

Meanwhile, AMPK is an enzyme which is activated under the condition lacking intracellular energy and helps recovery from the condition lacking energy by various functions of controlling metabolism. In the present invention, the effect of chito-oligosaccharide on AMPK activation in liver cells was investigated in vitro and in vivo tests. Activated AMPK inactivates ACC in liver cells, which decreases the concentration of ACC metabolites such as malonyl-CoA which is a potent inhibitor of carnithine palmitoyltransferase-1 (CPT-1) which plays a role in transport of fatty acids into mitochondria. In order to study it, the change of ACC by an addition of chito-oligosaccharide was determined. Further, as the activity of ACC is reduced, the capacity of inhibiting CPT-1 is decreased, and thus the oxidation of fatty acid can be promoted. Thus, the degree of oxidation of fatty acid according to the administration of chito-oligosaccharide was measured. Furthermore, AMPK is an energy-sensor enzyme and thus, when energy is depleted due to exercise and the ratio of ATP/AMP or phosphocreatin/creatine is decreased, AMPK makes the path of consumption of ATP intercepted and the path for the production of ATP opened. Based on it, the change of intracellular ATP content according to the increase of AMPK activity was investigated.

Furthermore, in order to test the effect of chito-oligosaccharide on the activation of energy metabolism in an animal, after feeding mice with chito-oligosaccharide, exercise behavior was observed. Also, indices in blood related to fatigue and liver function were measured.

### In Vitro Test

### Test Example 1

### Effect of Chito-Oligosaccharide on AMPK Activation

Chito-oligosaccharides with a molecular weight of 2,000 and 9,000 (100, 500 ppm) and liver cells (HepG2) treated by AICAR (1 mM) were added to a lysis buffer (50 mM Tris-HCl, pH 7.4, 150 mM NaCl, 0.1% SDS, 1.0% Triton X-100. 0.25% Deoxycholate, 1 mM EDTA, 1 mM PMSF) to disrupt the cells. The homogenate was centrifuged at 12,000 rpm for 15 minutes and a layer of protein was obtained. The degree of expression of ACC and p-ACC proteins was determined by a western blotting. 50 μg of protein was loaded on SDS-polyacrylamide gel, electrophoresis was carried out, and the gel was transferred to a nitrocellulose membrane. The membrane was reacted with blocking solution (5% skim milk, 10 mM Tris, pH7.5, 10 mM NaCl, 0.1% Tween 20) for 1 hour to remove nonspecific bindings, the primary antibodies to AMPK and p-AMPK proteins were added and it was maintained overnight at 4° C. Thereafter, it was washed with a blocking solution for 10 minutes three times, the secondary antibodies were added thereto, and they were reacted for 1 hour and washed as described above. After adding ECL solution, the membrane was exposed to X-ray film and the amount of protein was measured. The change in the amount of protein was calculated by an optical density with an image analyzer.

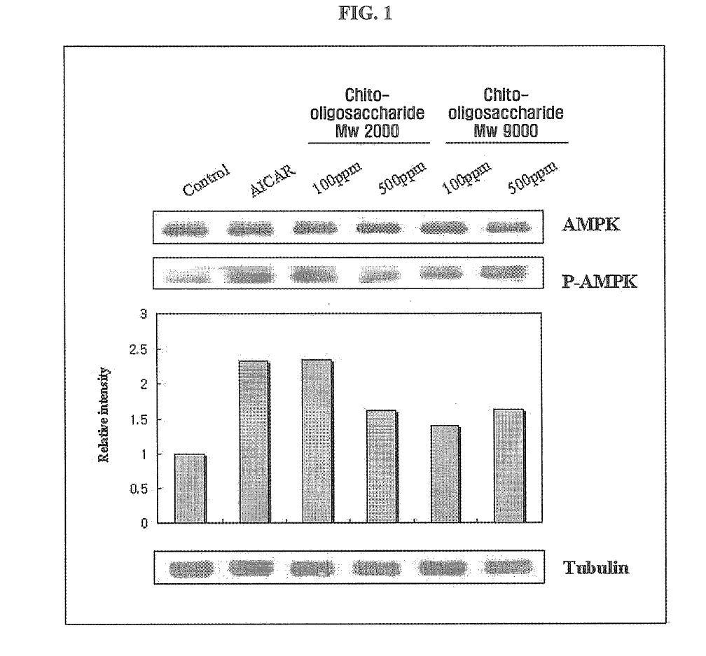

FIG. 1 is a result showing the change in the amount of AMPK protein expression when liver cells were treated with chito-oligosaccharide and cultured. It is shown that, when AICAR known as an AMPK activator was added, the expression of activated phosphor-AMPK was increased twice or more. The expression of p-AMPK protein was also increased by chito-oligosaccharide, and chito-oligosaccharide with a molecular weight 2,000 showed a similar activity to that of AICAR. There was no significant difference between the treated groups in the total amount of AMPK expression. From the above result, it can be understood that chito-oligosaccharide promotes the expression of AMPK.

### Test Example 2

### Effect of Chito-Oligosaccharide on ACC Activation

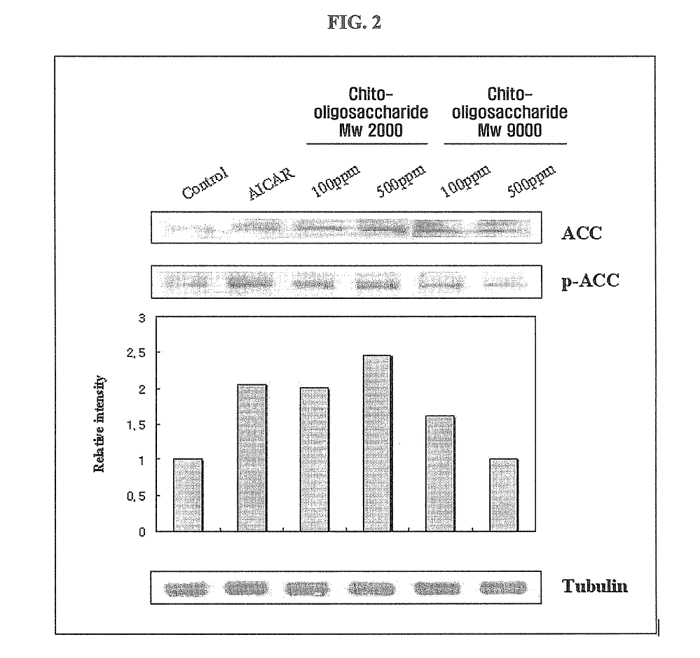

The expressions of ACC and p-ACC proteins were measured by a Western blotting as described above. As a result, as shown in FIG. 2, the total amount of ACC protein expression was not changed by the treatment of AICAR and chito-oligosaccharide, but the amount of p-ACC protein expression was distinctly changed by them. AICAR increased the amount of p-ACC expression in liver cells up to twice and the group treated by chito-oligosaccharide also showed the increased expression of p-ACC. Chito-oligosaccharide with a molecular weight of 2000 increased the expression of p-ACC depending on the concentration of it, and thus 100 ppm of the chito-oligonucleotide showed an activity similar to AICAR and 500 ppm of chito-oligosaccharide provided an activity higher than AICAR. Chito-oligosaccharide with a molecular weight of 9,000 showed an activity lower than chito-oligosaccharide with a molecular weight of 2,000, and 100 ppm of the chito-oligosaccharide increased the expression of p-ACC up to 1.5 times.

From this result, it can be understood that AICAR and chito-oligosaccharide increase the expression of p-ACC which is an inactivated form of ACC, and thus inhibit the conversion of acetyl-CoA into malonyl-CoA. This suggests that chito-oligosaccharide increases the activity of AMPK in liver cells and inhibits the activity of ACC which is regulated by AMPK, and thus can inhibit the synthesis of fatty acids.

### Test Example 3

### Effect of Chito-Oligosaccharide on Fatty Acid Oxidation

To determine the degree of fatty acid oxidation in liver cells, HepG2 cells were seeded on a 96-well plate. After stabilizing the seeded plate, AICAR and chito-oligosaccharide were added in a concentration of 100 and 500 ppm. After culturing for 6 hours, to the cells was added 7 ml of assay buffer (20 mM HEPES, 25 mM NaHCO₃, 1.2 mM KH₂PO₄, 3 mM glucose, 114 mM NaCl, 4.7 mM KCl, 1.2 mM MgSO₄, 2.5 mM CaCl₂, 1% ultra fatty acid-free BSA) and they are reacted for 30 minutes. Then, [1-¹⁴C]-palmitate (3.4 μCi; 1.0 μCi/μmole) was incorporated thereto and labeling was carried out for 2 hours. 5% perchloric acid was added to stop the reaction and the amount of radiation was measured.

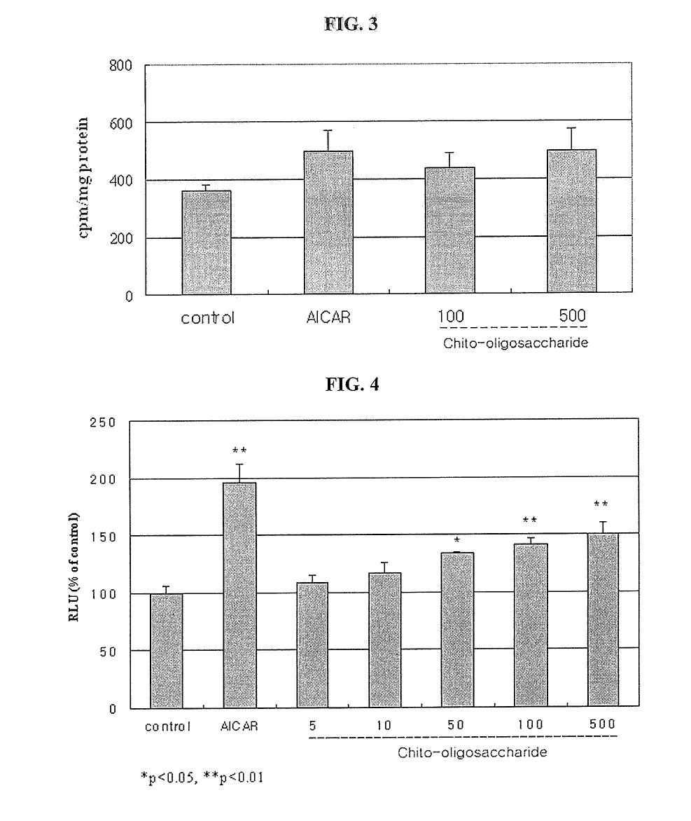

As shown in FIG. 3, the positive control group treated with AICAR showed about 35% increased oxidation of fatty acid and the group treated with chito-oligosaccharide increased the oxidation of fatty acid depending on the concentration of chito-oligosaccharide. It can be understood that the group treated with 500 ppm of chito-oligosaccharide promotes the oxidation similar to the group treated with AICAR. In view of the fact that ACC and malonyl-CoA which is produced by ACC were known as inhibiting the activity of CPT-1 which is an important enzyme involved in fatty acid oxidation, it can be seen that the expression of p-ACC which is an inactivated form of ACC was increased by the addition of chito-oligosaccharide, and thus the inhibitory activity to CPT-1 was decreased, resulting in the promotion of fatty acid oxidation.

### Test Example 4

### Effect of Chito-Oligosaccharide on the Production of ATP

The production of ATP according to AMPK activation was measured by ATP assay kit. HepG2 cells were seeded on a 96-well plate and the seeded plate was stabilized and, thereafter, AICAR and chito-oligosaccharide were added in a concentration of 5 to 500 ppm. After culturing for 24 hours, the reagent prepared by mixing a substrate and a buffer was added to each well and they were reacted for 30 minutes at a room temperature. Cells were disrupted by mixing them in an orbital shaker and, after stabilizing them for 10 minutes, a luminescence was measured.

As a result, AICAR known as an AMPK activator increased the production of ATP up to twice. The production of ATP was also increased by chito-oligosaccharide depending on the concentration of it and, when they were treated by 500 ppm of chito-oligosaccharide, the production of ATP was increased up to 1.5 times, which indicates the activation of energy metabolism by chito-oligosaccharide in liver cells.

### In Vivo Test

### Reference Example 1

### Test Animal and Method

Hairless mice at age of three weeks were purchased and divided into 10 mice/cage. To minimize the effect by mouse hair in a forced swimming test, hairless mice were used. They could be freely intake feeds and water, 22±1° C. of temperature and 60±5% of moisture were maintained, and dark and bright was changed in an interval of 12 hours. The experimental animals were divided into exercise and non-exercise groups (see Table 1) and the change according to feeds alone or feeds followed by exercise was analyzed.

TABLE 1
Constitution of test groups

| | | | The |
|---|---|---|---|
| | | | number of |
| Test group | | Test material | animals |
| non- | control group | — | 10 |
| exercise | test group 1 | chito- | 10 |
| Group | | oligosaccharide | |
| | | 2000 | |
| | test group 2 | chito- | 10 |
| | | oligosaccharide 9000 | |
| Exercise | control group | — | 10 |
| Group | test group 1 | chito- | 10 |
| | | oligosaccharide 2000 | |
| | test group 2 | chito- | 10 |
| | | oligosaccharide 9000 | |

As for the test groups, feeds comprising the test material were given for 4 weeks. As for the non-exercise groups, blood and tissue were analyzed after 4 weeks of administration and as for the exercise groups, mice were trained for adaptation by swimming exercise (twice/week) for 4 weeks of administration. After 4 weeks of administration, a forced swimming test was performed and blood and tissue prepared after the forced swimming were analyzed.

### Reference Example 2

### Composition of Feeds

The test substance of chito-oligosaccharide was comprised in feeds in an amount of 0.5%. For 4 weeks conventional feeds/test feeds were provided. The composition of feeds is shown in Table 2 and the weight difference caused by the addition of chito-oligosaccharide was regulated by the addition of corn starch.

TABLE 2
Composition of feeds

Test group

| Ingredient | Control group | MW 2000 | MW 9000 |
|---|---|---|---|
| Casein | 200 | 200 | 200 |
| Corn starch | 397.486 | 392.486 | 392.486 |
| Sucrose | 100 | 100 | 100 |
| Dextrose | 132 | 132 | 132 |
| Cellulose | 50 | 50 | 50 |
| Soybean oil | 70 | 70 | 70 |
| Mineral mixture | 35 | 35 | 35 |
| Vitamin mixture | 10 | 10 | 10 |
| TBHQ | 0.014 | 0.014 | 0.014 |
| L-cystein | 3 | 3 | 3 |
| Choline bitartrate | 2.5 | 2.5 | 2.5 |
| Chito- | 0 | 5 | 5 |
| oligosaccharide | | | |

### Test Example 5

### Anti-Fatigue Effect on the Mice in a Exercise Group—Forced Swimming Test (FST)

FST is a method usually used in an animal to determine the level of depression and has been utilized in a pre-clinical test. Also, the test has been applied to a method to prove the effect of certain materials on anti-fatigue and endurance (Koo H N et al., Biol Pharm Bull, 27, 117-119, 2004; Shin H Y et al., Biol Pharm Bull, 27, 1521-1526, 2004).

In this FST, the duration of immobility of mouse for 6 minutes was measured. Two opaque glass cylinders (height: 25 cm; diameter: 10 cm) were filled with water up to the height of 10 cm and two mice were subjected to the test. After 2 minutes for stabilization, the duration of immobility was recorded during the last 4 minutes. A mouse was considered immobile when floating with the head above the surface of water without active movement.

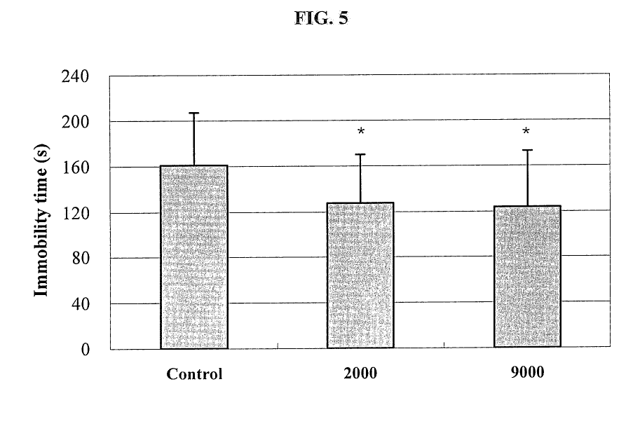

As shown in FIG. 5, the duration of immobility was significantly reduced in the chito-oligosaccharide-treated group (128±42 s, 124±49 s), compared with the control group (161±46 s). The effect in the group treated with chito-oligosaccharide having a molecular weight of 2000 was similar to that in the group treated with chito-oligosaccharide having a molecular weight of 9000. This suggests that the administration of chito-oligosaccharide affect fatigue-related metabolism and biosystem in a mouse.

### Test Example 6

### Change in Blood Indices

During 4 weeks fed with chito-oligosaccharide, mice were adapted to swimming for 10 minutes (twice/week) on the 2^{nd}, 3^{rd}, and 4^{th} week. After 4 weeks, mice were forced to swim for 80 minutes under the same condition and then subjected to euthanasia to bleed. In a non-exercise group, mice were blooded immediately after 4 weeks feeding. It was known that enzymes in blood such as LDH, etc., are changed from the stress caused by excessive exercise and the increase of corticosteroid in blood by stress affects a lipid metabolism. Based on this understanding, after isolating serum from blood, GOT, GPT, LDH, free fatty acid and total cholesterol were measured. The results are shown in Tables 3 and 4.

TABLE 3
Blood analysis in non-exercise groups

| | | | | | Total |
|---|---|---|---|---|---|
| Serum | GOT | GPT | LDH | FFA | cholesterol |
| Non- | 161.9 ± 39.4 | 145.7 ± 75.7 | 964.7 ± 189.9 | 4396.7 ± 827.8 | 153.7 ± 21.2 |
| exercise | | | | | |
| group | | | | | |
| Non- | 186.3 ± 74.3 | 164.5 ± 82.1 | 923.3 ± 198.3 | 4396.5 ± 690.1 | 168.2 ± 14.8 |
| exercise | | | | | |
| group - | | | | | |
| chito 2000 | | | | | |
| Non- | 166.3 ± 23.6 | 122.2 ± 99.7 | 908.0 ± 507.0 | 4946.1 ± 265.4 | 176.5 ± 30.7 |
| exercise | | | | | |
| group - | | | | | |
| chito 9000 | | | | | |

TABLE 4
Blood analysis in exercise groups

| | | | | | Total |
|---|---|---|---|---|---|
| Serum | GOT | GPT | LDH | FFA | cholesterol |
| Exercise group | 221.9 ± 152.2 | 169.6 ± 184.0 | 1312.5 ± 489.9 | 3664.7 ± 33.5 | 159.5 ± 14.7 |
| Exercise group - | 233.9 ± 167.2 | 93.1 ± 79.4 | 1396.1 ± 608.7 | 3564.8 ± 177.0 | 139.8 ± 36.6 |
| chito 2000 | | | | | |
| Exercise group - | 189.0 ± 69.09 | 80.2 ± 30.0 | 1193.8 ± 273.9 | 2899.0 ± 251.6* | 114.2 ± 15.4* |
| chito 9000 | | | | | |

As shown in Table 3, in the chito-oligosaccharide-administered non-exercise groups, there was a tendency that the level of GPT and LDH in serum is decreased. When mice were forced to swim after chito-oligosaccharide feeding, there was a tendency that the levels of GOT and LDH is increased, compared with that in the non-exercise control group. However, in the chito-oligosaccharide 9000-administered group, the derivation between individuals in the levels of GOT, GPT and LDH was decreased and the average values were reduced to the normal levels. The value of fatty acid in blood was significantly reduced from the energy consumption by exercise. Further, in the chito-oligosaccharide-9000 administered group, the levels of fatty acid and cholesterol was significantly reduced, compared with that in the control group. This indicates that chito-oligosaccharide is effective for improving the metabolism efficiency of lipid in blood and inhibiting fatigue caused by exercise and suggests that the chito-oligosaccharide with a molecular weight of 9000 is more effective.

### INDUSTRIAL APPLICABILITY

The production of energy is increased by AMPK activation, and thus the increased energy production under the circumstance requiring energy such as exercise or in everyday life makes exercise capacity improved and fatigues reduced. Therefore, a composition for promoting AMPK activity, which comprises a chito-oligosaccharide can be developed to provide food or medicinal products as an endurance builder or for the prevention and improvement of fatigue.

1. A composition for promoting AMPK activity, which comprises a chito-oligosaccharide as an active ingredient.

2. The composition of claim 1, wherein the chito-oligosaccharide is comprised in an amount of 10 to 90% by weight based on the total weight of the composition.

3. The composition of claim 1, wherein the chito-oligosaccharide has a molecular weight of 700 to 9,000.

4. The composition of claim 1, wherein the composition inhibits ACC activity and promotes the oxidation of fatty acids.

5. The composition of claim 1, wherein the composition enhances energy metabolism in liver cells.

6. A health food composition which comprises the composition as claimed in claim 1.

7. Use of chito-oligosaccharide-containing composition for promoting AMPK activity.

8. Use of chito-oligosaccharide-containing composition for improving fatigue recovery.
